SEQUENCE LISTING (14 sequences)

SEQ ID NO: 1 - DNA, 1087 bp, Lactococcus lactis subsp. cremoris A17
  gene (1)..(1087)
gggggggggggtgctatacatgcagttgagcgatgaagattggtgcttgcaccaatttgaagagcagcgaacgggtgagtaacgcgtggggaatctgcctttgagcgggggacaacatttggaaacgaatgctaataccgcataacaactttaaacacaagttttaagtttgaaagatgcaattgcatcactcaaagatgatcccgcgttgtattagctagttggtgaggtaaaggctcaccaaggcgatgatacatagccgacctgagagggtgatcggccacattgggactgagacacggcccaaactcctacgggaggcagcagtagggaatcttcggcaatggacgaaagtctgaccgagcaacgccgcgtgagtgaagaaggttttcggatcgtaaaactctgttggtagagaagaacgttggtgagagtggaaagctcatcaagtgacggtaactacccagaaagggacggctaactacgtgccagcagccgcggtaatacgtaggtcccgagcgttgtccggatttattgggcgtaaagcgagcgcaggtggtttattaagtctggtgtaaaaggcagtggctcaaccattgtatgcattggaaactggtagacttgagtgcaggagaggagagtggaattccatgtgtagcggtgaaatgcgtagatatatggaggaacaccggtggcgaaagcggctctctggcctgtaactgacactgaggctcgaaagcgtggggagcaaacaggattagataccctggtagtccacgccgtaaacgatgagtgctagatgtagggagctataagttctctgtatcgcagctaacgcaataagcactccgcctggggagtacgaccgcaaggttgaaactcaaaggaattgacgggggcccgcacaagcggtggagcatgtggtttaattcgaagcaacgcgagaaccttaccaggtcttgacatactcgtgctattcctagagataggaagttccttcgggacacgggatacagtggtgcatggttgtcgtcagctcgtgtcgtgagatgttgggttaagtcccgcaacgagcgccaccccctat

SEQ ID NO: 2 - DNA, 10 bp, Artificial
  primer
ccgcagccaa

SEQ ID NO: 3 - DNA, 10 bp, Artificial
  primer
aacgcgcaac

SEQ ID NO: 4 - DNA, 10 bp, Artificial
  primer
gcggaaatag

SEQ ID NO: 5 - DNA, 21 bp, Artificial
  Forward primer
gctggagaactctgacccgcc

SEQ ID NO: 6 - DNA, 20 bp, Artificial
  Reverse primer
caaggatggccgcgtcgttg

SEQ ID NO: 7 - DNA, 20 bp, Artificial
  Forward primer
aggagtgccccgctttcacc

SEQ ID NO: 8 - DNA, 19 bp, Artificial
  Reverse primer
tgccagagcggctgccaga

SEQ ID NO: 9 - DNA, 20 bp, Artificial
  Forward primer
agcagaacaccacactgaca

SEQ ID NO: 10 - DNA, 18 bp, Artificial
  Reverse primer
ccttggctgtgatgcgat

SEQ ID NO: 11 - DNA, 20 bp, Artificial
  Forward primer
cagggactcaagagcaacac

SEQ ID NO: 12 - DNA, 20 bp, Artificial
  Reverse primer
gctgagccactttaggttct

SEQ ID NO: 13 - DNA, 22 bp, Artificial
  Forward primer
gtatgactccactcacggcaaa

SEQ ID NO: 14 - DNA, 20 bp, Artificial
  Reverse primer
ggtctcgctcctggaagatg

(19) United States
(12) Patent Application Publication
(10) Pub. No.: US 2015/0017208 A1
(43) Pub. Date: Jan. 15, 2015

(54) LACTIC ACID BACTERIUM HAVING IMMUNOMODULATORY AND ANTI-ALLERGIC EFFECTS

(71) Applicant: National Yang-Ming University, Taipei (TW)

(72) Inventors: Ying-Chieh Tsai, Taipei (TW); Hui-Ching Mei, Taipei (TW); Yen-Wenn Liu, Taipei (TW); Wan-Yu Chen, Taipei (TW); Chien-Chen Wu, Taipei (TW)

(21) Appl. No.: 13/940,620

(22) Filed: Jul. 12, 2013

(51) Int. Cl. A61K 35/74

(52) U.S. Cl. CPC A61K 35/74
USPC 424/234.1; 435/252.1

(57) ABSTRACT

A novel lactic acid bacterium strain having immunomodulatory and anti-allergic effects in a subject is disclosed. Lactococcus lactis subsp. cremoris A17 deposited under DSMZ Accession No. DSM 27109 is disclosed. A composition including the novel lactic acid bacterium strain and a method for using the lactic acid bacterium strain are also disclosed.

### BACKGROUND OF THE INVENTION

1. Field of the Invention

The present invention relates to a lactic acid bacterium, and more particularly relates to a novel lactic acid bacterium strain having immunomodulatory and anti-allergic effects in a subject.

2. Description of Related Art

Lactic acid bacteria (LAB) are generally believed to promote human health. Various beneficial effects of LAB have been reported in the treatment of inflammatory disorders like ulcerative colitis, the maintenance of intestinal homeostasis, and the amelioration of atopic dermatitis in infants. Nevertheless, the effectiveness of LAB is variable due to the use of different strains.

Allergic disorders, such as allergic rhinitis, atopic dermatitis, allergic asthma, and food allergies, have become increasingly prevalent in many countries. Allergies are related to the T-helper cell type 2 (Th2) responses both in T-cells and B-cells. Th2 responses are characterized by the production of certain cytokines including interleukin (IL)-4, IL-5, IL-13, and the production of total immunoglobulin (Ig) E, antigen-specific IgE and IgG1. Cytokine production is regarded as T-cell response, and immunoglobulin production is regarded as B-cell response. Th1 cells can suppress Th2 responses by secreting interferon (IFN)-γ, IgG2a, IL-2, and IL-3. Therefore, to regulate the immune responses by suppressing the Th2-response and enhancing the Th1-response is expected to be helpful in the treatment of allergy and other Th2 dominant disorders and maintaining healthy immune condition.

Toll-like receptors (TLRs) and nucleotide-binding oligomerization domain protein (NOD)-like receptors (NLRs) are receptors that detect unique bacterial components and subsequently activate immune responses in a host. Oral administration of LAB might trigger the immune responses via these receptors. TLRs and NODs comprise a family of pattern-recognition receptors that are known to respond to microbial specific patterns. Recently, the expression of nucleotide oligomerization domain 1 (NOD-1) and NOD-2 (both belong to the NLRs family) is proved to be necessary for Th2 priming, including T cell and B cell responses. NOD-2 is shown to break tolerance to inhaled antigen. This suggests the potential of NOD-2 in driving Th2 lung inflammation. TLR-4 signaling is also reported to be required for Th2 priming to antigen.

Numerous studies have proposed that LAB, live or heat-killed, alleviate allergic symptoms by modulating the Th1/Th2 balance toward a Th1 dominant state. Perinatal administration of live Lactobacillus rhamnosus GG (LGG) reduces the development of eczema in children with a family history of this atopic disorder. Heat-killed Lactobacillus casei strain Shirota (LcS) stimulates IL-12 secretion, which shifts the cytokine production pattern from a Th2 to Th1 predominance and thereby suppresses IgE production, IgG1 responses, and systemic anaphylaxis.

### SUMMARY OF THE INVENTION

The present invention relates to a novel lactic acid bacterium, which is Lactococcus lactis subsp. cremoris.

Lactococcus lactis subsp. cremoris A17 (abbreviated as A17 hereafter) has been deposited under Budapest Treaty at DSMZ-DEUTSCHE SAMMLUNG VON MIKROORGANISMEN UND ZELLKULTUREN GmbH (Inhoffenstr. 7 B, D-38124 Braunschweig, Germany) on Apr. 11, 2013 and has been given the DSMZ Accession No. DSM 27109 by the International Depositary Authority. This biological material was subjected to the viability test and passed. In a further aspect of the present invention, the lactic acid bacterium may be heat-inactivated.

Further, a composition is provided and includes a lactic acid bacterium, which is Lactococcus lactis subsp. cremoris. In a further aspect, the lactic acid bacterium may be Lactococcus lactis subsp. cremoris A17 deposited under DSMZ Accession No. DSM 27109. In a still further aspect, the lactic acid bacterium may be heat-inactivated.

The present invention further provides a method for treating or preventing a disorder that includes the step of administering an effective amount of a lactic acid bacterium to a subject. Further, in the method of the present invention, the lactic acid bacterium may be Lactococcus lactis subsp. cremoris A17 deposited under DSMZ Accession No. DSM 27109. In the method of the present invention, the lactic acid bacterium may be heat-inactivated.

In accordance with the present invention, a disorder is related to expression of a protein selected from the group consisting of IgG1, IgG2a, IgE, IFN-γ, IL-4, NOD-1, NOD-2 and TLR-4. Furthermore, in one embodiment of the present invention, expression of IgG2a or IFN-γ is increased, expression of IgG1, IgE or IL-4 is decreased, or mRNA expression of NOD-1, NOD-2 or TLR-4 is down-regulated.

In one embodiment, a disorder is an allergic disorder. In one embodiment, the allergic disorder is allergic rhinitis, atopic dermatitis, allergic asthma or a food allergy.

In one embodiment, a lactic acid bacterium is orally administrated for treating or preventing a disorder.

The present invention further provides a method for modulating an immune response that includes the step of administering an effective amount of a lactic acid bacterium to a subject. Further, in the method of the present invention, the lactic acid bacterium is Lactococcus lactis subsp. cremoris A17 deposited under DSMZ Accession No. DSM 27109. In one embodiment, the lactic acid bacterium may be heat-inactivated.

In accordance with the present invention, an immune response is related to expression of a protein selected from the group consisting of IgG1, IgG2a, IgE, IFN-γ, IL-4, NOD-1, NOD-2 and TLR-4. Furthermore, in one embodiment of the present invention, expression of IgG2a or IFN-γ is increased, expression of IgG1, IgE or IL-4 is decreased, or mRNA expression of NOD-1, NOD-2 or TLR-4 is down-regulated.

In one embodiment, an immune response may be related to an allergic disorder. In one embodiment of the present invention, the disorder may be an allergic disorder. In another embodiment, the allergic disorder may be allergic rhinitis, atopic dermatitis, allergic asthma or a food allergy.

In one embodiment, a lactic acid bacterium may be orally administrated for modulating an immune response.

### BRIEF DESCRIPTION OF THE DRAWINGS

FIG. 1(A) to FIG. 1(C) show a set of electrophoresis photographs showing the RAPD profiles of seven Lactococcus lactis strains;

FIG. 2 shows IFN-γ productions by human peripheral blood mononuclear cells (hPBMCs) stimulated with heat-killed Lactococcus lactis subsp. cremoris A17 (A17), Lactobacillus casei strain Shirota (LcS) and Lactobacillus rhamnosus GG (LGG);

FIG. 3 shows an experimental timeline for the ovalbimin (OVA)-sensitized BALB/c mouse model;

FIG. 4(a) to FIG. 4(d) show the effects of oral administration of live or heat-killed Lactococcus lactis subsp. cremoris A17 (A17) on immunoglobulins production in OVA-sensitized mouse serum;

FIG. 5(a) to FIG. 5(d) show the effect of oral administration of live or heat-killed Lactococcus lactis subsp. cremoris A17 (A17) on cytokine production in OVA-sensitized mouse spleen cell cultures; and

FIG. 6(a) to FIG. (d) show the effect of oral administration of live or heat-killed Lactococcus lactis subsp. cremoris A17 (A17) on mRNA expression in OVA-sensitized mouse spleen cells.

### DETAILED DESCRIPTION OF THE PREFERRED EMBODIMENTS

The following illustrative embodiments are provided to illustrate the disclosure of the present invention. These and other advantages and effects can be apparently understood by those in the art after reading the disclosure of this specification.

### Example 1

### Isolation and Genetic Typing of Lactococcus lactis Subsp. cremoris A17

Lactococcus lactis subsp. cremoris A17 was isolated from Taiwanese fermented cabbage. 16S rDNA from A17 (SEQ ID NO. 1) was analyzed by direct sequencing of about 1000 nucleotides of PCR-amplified 16S rDNA. Genomic DNA extraction, PCR mediated amplification of the 16S rDNA, purification of the PCR product, and sequencing of the purified PCR product were carried out, accordingly.

The resulting sequence was put into the alignment software provided online by the National Center for Biotechnology Information (NCBI) (http://www.ncbi.nlm.nih.gov/), aligned manually and compared with representative 16S rDNA sequences of organisms belonging to the Firmicutes. For comparison, 16S rDNA sequences were also obtained from the database provided online by the NCBI.

As a result of this analysis, the following Table 1 lists those organisms, whose 16S rDNA sequences show the highest similarity values compared to the 16S rDNA sequence of Lactococcus lactis subsp. cremoris A17.

TABLE 1
Comparison Between 16S rDNA Sequences

| | % 16S rRNA gene |
|---|---|
| | sequence |
| Strain (GenBank accession number) | similarity to A17 |
| Lactococcus lactis subsp. cremoris NCDO607^{T} | 99.8 |
| (AB100802) | |
| Lactococcus lactis subsp. cremoris (M58836) | 99.1 |
| Lactococcus lactis subsp. lactis NCDO604^{T} | 98.8 |
| (AB100803) | |
| Lactococcus lactis subsp. hordniae NCDO2181^{T} | 98.8 |
| (AB100804) | |
| Lactococcus plantarum (X54259) | 90.2 |
| Lactococcus garvieae (X54262) | 89.1 |
| Lactococcus piscium (X53905) | 88.3 |
| Lactococcus raffinolactis (X54261) | 87.9 |

The partial 16S rDNA sequence of A17 shows highest similarity to Lactococcus lactis subsp. cremoris. Consequently, A17 represents a strain of Lactococcus lactis subsp. cremoris, but also represents a new species within the genus Lactococcus.

Lactococcus lactis subsp. cremoris A17 has been deposited under Budapest Treaty at DSMZ-DEUTSCHE SAMMLUNG VON MIKROORGANISMEN UND ZELLKULTUREN GmbH (Inhoffenstr. 7 B, D-38124 Braunschweig, Germany) on Apr. 11, 2013 and has been given the DSMZ Accession No. DSM 27109 by the International Depositary Authority. This biological material was subjected to the viability test and passed.

### Example 2

### Identification of the Bacterial Strains Using RAPD-PCR

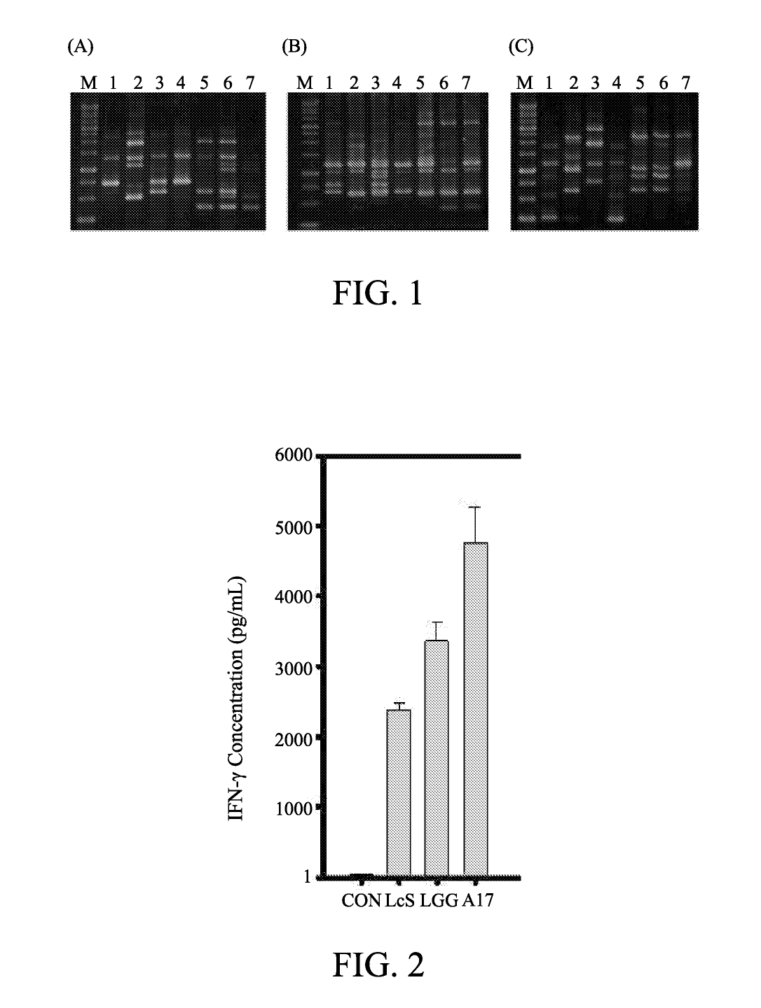

The RAPD profiles of A17 and other six Lactococcus lactis strains were compared. PCR was carried out under the condition indicated in Table 2 using the random primer 1254 (5′-CCGCAGCCAA-3′, SEQ ID NO. 2), 1281 (5′-AACGCGCAAC-3′, SEQ ID NO. 3), or 1252 (5′-GCGGAAATAG-3′, SEQ ID NO. 4). DNAs respectively extracted from these strains were used as templates. The obtained amplification products were electrophoresed and the patterns were compared as shown in FIG. 1(A) to FIG. 1 (C).

TABLE 2
Composition of the PCR reaction solution (25 μl)

| Component | Volume |
|---|---|
| ddH₂O | 17.9 μl |
| 10X PCR Buffer | 2.5 μl |
| dNTP Mix (2.5 mM) | 2.0 μl |
| MgCl₂ (25 mM) | 1.0 μl |
| primer | 0.4 μl |
| rTaq | 0.2 μl |
| DNA template (10 μM) | 1.0 μl |

### PCR Conditions:

94° C., 2 min; 5 cycles (94° C., 30 sec.; 36° C., 1 min; 72° C., 1.5 min.); 30 cycles (94° C., 20 sec.; 36° C., 30 sec.; 72° C., 1.5 min); 72° C., 3 min.

As shown in FIG. 1(A) to FIG. 1(C), Lane M represents DNA ladder (250-10000 bp); Lane 1 represents ATCC 19257; Lane 2 represents ATCC 19435; Lane 3 represents A17; Lane 4 represents BCRC 12304; Lane 5 represents ATCC 11454; Lane 6 represents BCRC 12315; and Lane 7 represents ATCC 13675. The result indicated that the amplification products of Lactococcus lactis subsp. cremoris A17 had different patterns from other six Lactococcus lactis strains.

### Example 3

### API Typing

Sugar utilization of A17 of the present invention isolated above was investigated using API50CHL kit (bioMerieux, France), and the results are shown in Table 3.

TABLE 3
Results of Fermentation Test^{a}

| carbohydrates | | ATCC | ATCC | carbohydrates | | ATCC | ATCC |
|---|---|---|---|---|---|---|---|
| substrate | A17 | 19435^{b} | 19257^{c} | substrate | A17 | 19435^{b} | 19257^{c} |
| CONTROL | − | − | − | Esculin ferric | + | + | + |
| | | | | citrate | | | |
| Glycerol | − | − | − | Salicin | + | + | − |
| Erythritol | − | − | − | D-Cellobiose | + | + | + |
| D-Arabinose | − | − | − | D-Maltose | + | + | − |
| L-Arabinose | − | − | − | D-Lactose | + | − | + |
| | | | | (bovine origin) | | | |
| D-Ribose | + | + | − | D-Melibiose | − | − | − |
| D-Xylose | + | + | − | D-Saccharose | + | − | − |
| | | | | (sucrose) | | | |
| L-Xylose | − | − | − | D-Trehalose | + | + | − |
| D-Adonitol | − | − | − | Inulin | − | − | − |
| Methyl-β-D- | − | − | − | D-Melezitose | − | − | − |
| Xylopyranoside | | | | | | | |
| D-Galactose | + | + | + | D-Raffinose | − | − | − |
| D-Glucose | + | + | + | Amidon (starch) | + | + | − |
| D-Fructose | + | + | + | Glycogen | − | − | − |
| D-Mannose | + | + | + | Xylitol | − | − | − |
| L-Sorbose | − | − | − | Gentiobiose | + | + | − |
| L-Rhamnose | − | − | − | D-Turanose | − | − | − |
| Dulcitol | − | − | − | D-Lyxose | weak | − | − |
| Inositol | − | − | − | D-Tagatose | − | − | − |
| D-Mannitol | + | − | − | D-Fucose | − | − | − |
| D-Sorbitol | − | − | − | L-Fucose | − | − | − |
| Methyl-α-D- | − | − | − | D-Arabitol | − | − | − |
| mannopyranoside | | | | | | | |
| Methyl-α-D- | − | − | − | L-Arabitol | − | − | − |
| glucopyranoside | | | | | | | |
| N-Acetyl | + | + | + | Potassium gluconate | + | − | − |
| glucosamine | | | | | | | |
| Amygdalin | + | + | − | Potassium 2- | − | − | − |
| | | | | ketogluconate | | | |
| Arbutin | + | + | − | Potassium 5- | − | − | − |
| | | | | ketogluconate | | | |

^{a}+, positive; −, negative
^{b}ATCC 19435, Lactococcus lactis subsp. lactis type strain
^{c}ATCC 19257, Lactococcus lactis subsp. cremoris type strain

Although A17 was classified as Lactococcus lactis subsp. cremoris based on the comparison of 16S rDNA sequences, it harbored a biochemical property similar to Lactococcus lactis subsp. lactis, especially the inability to produce acid from maltose and ribose, which is generally used for the differentiation of subsp. lactis and cremoris. Moreover, compared to the type strains of Lactococcus lactis subsp. lactis ATCC 19435 and Lactococcus lactis subsp. cremoris ATCC 19257, A17 was able to produce acid from saccharose, mannitol, and potassium gluconate, implying a particular biochemical property of A17.

### Example 4

### Preparation of Lactococcus lactis Subsp. cremoris A17

Lactococcus lactis subsp. cremoris A17 was inoculated in de Man, Rogosa, and Sharpe (MRS, pH 5.4; Difco, USA) broth, cultured at 30° C. for 48 h. For a live A17 preparation, pelleted bacteria were washed twice with sterile phosphate buffered saline (PBS) and then resuspended to a final concentration of 10¹⁰ CFU/mL in PBS. As for a heat-killed A17 preparation, 10¹⁰ CFU/mL of A17 were heat-killed at 100° C. for 20 min as experimentally required and were stored at −20° C. until use.

### Example 5

### Preparation and Stimulation of Human Peripheral Blood Mononuclear Cell

hPBMCs were isolated from healthy volunteers with no history of atopic disorder. In brief, hPBMCs were isolated by centrifugation at 1,500 rpm for 20 mins using Ficoll (GE, Uppsala, Sweden). After washing, the hPBMCs were harvested and resuspended in RPMI 1640 culture medium supplemented with 10% FBS, 1% L-glutamate, 100 IU/ml penicillin, 0.1 mg/ml streptomycin, and 0.25 μg/ml amphotericin.

The effects of LAB on hPBMC cytokine production were used to evaluate the in vitro immunomodulatory activities of LAB. Cell cultures were set up in triplicate in 96-well flat bottom polystyrene microtitre plates. All cultures contained 1×10⁵ cells of hPBMCs and 5×10⁷ CFU of heat-killed LAB. Heat-killed LGG and LcS were used as positive controls. The plates were incubated at 37° C. in 5% CO₂. The supernatants from the cultures were collected at 48 h and stored at −20° C. until used for cytokine analysis. Cell viability was measured by using an MTT assay. A17 that had a corresponding hPBMC viability exceeding 90% was selected for further cytokine measurement.

The in vitro immunological effects of LAB strains on hPBMCs were subsequently evaluated. The immunological effects of LAB on hPBMCs were evaluated by measuring the levels of the cytokine IFN-γ, which is generally considered to be a Th1 cytokine. LGG and LcS, which are commercially available probiotics with recognized immunomodulatory function, were used as positive controls in this assay. A17, LcS, and LGG, were individually cultured with hPBMCs for determination of IFN-γ production.

FIG. 2 shows the effects of LcS, LGG, and Lactococcus lactis A17 on the production of IFN-γ. According to the results, LcS and LGG groups showing relatively high levels of IFN-γ indicated a Th1 dominant response. A17 stimulated the highest level of IFN-γ in comparison with those of LcS and LGG was further investigated its in vivo immunomodulatory activity.

### Example 6

### Experimental Animals and Feeds

Four-week-old female BALB/c mice were purchased from the National Laboratory Animal Center, Taiwan, and maintained in National Yang-Ming University. The animal room was kept on a 12 h light and dark cycle at 25±2° C. and 55±15% humidity. The mice were fed a standard laboratory diet (LabDiet Autoclavable Rodent Diet 5010, PMI Nutrition International, Brentwood, USA) to acclimate them for two weeks prior to bacterial feeding. All animal experimental procedures were reviewed and approved by the Animal Management Committee, National Yang-Ming University.

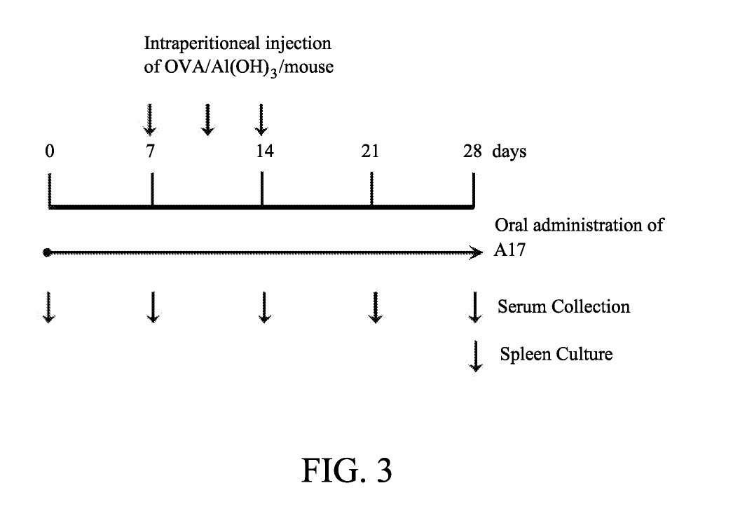

To evaluate the effect of A17 on immune responses, the 6-week-old mice were sensitized and challenged with OVA to establish an OVA-sensitized BALB/c mice model. The experimental procedure for immunization, administration of A17, and sample collection in the OVA-sensitized BALB/c mice model is summarized in FIG. 3. Four groups (n=8 in each group) of mice were assigned a different bacteria supplement for four consecutive weeks. The healthy control (CON group) and allergy control (OVA group) groups were orally administered PBS by stainless feeding tube. The other experimental groups were orally administered with either live A17 (10⁹ CFU/mouse/day, referred to A17-A) or heat-killed A17 (10⁹ CFU/mouse/day, referred to A17-H) by stainless feeding tube. All groups except for the healthy control group were intraperitoneally injected with 100 μl of Al(OH)₃ containing 50 μg of OVA three times on day 7, 11 and 14. The healthy control mice received Al(OH)₃ only. The body weights of all mice were measured every day during the study period. There were no significant differences in food intake, feed efficiency, or changes in body weight among the groups. Blood was collected by using retro-orbital venous plexus puncture and serum was prepared by centrifugation (2,000 rpm for 10 min) weekly starting from day 1 of the experiment. The serum was stored at −20° C. before immunoglobulin analysis.

### Example 7

### Preparation of Spleen Cells

Mice were sacrificed on day 28 and the spleen cells were harvested for culture. The spleen was ground with sterile flat bottom of a syringe piston to homogenize the spleen cells. The cells were adjusted to 1×10⁶ cells/ml in RPMI 1640 medium. In 24-well plates, cells were plated with or without mitogens, such as lipopolysaccharide (LPS, 600 ng/ml) or OVA (25 μg/m). The plates were incubated in a humidified incubator at 37° C. with 5% CO₂ for 48 hrs. After incubation, the supernatants were collected and stored at −20° C. for further cytokine analysis.

### Example 8

### Measurement of Immunoglobulins and Cytokines by an Enzyme-Linked Immunosorbent Assay (ELISA)

The levels of total IgE and OVA-specific IgE, IgG1, and IgG2a were measured by using the commercial ELISA kits (Bethyl Laboratory, Inc., Montgomery, USA). The concentrations of IFN-γ, IL-2, IL-4 and IL-10 were also determined by using ELISA procedure according to the manufacturers' instructions (for mouse cytokine determination, eBioscience, Boston, Mass.; for human cytokine measurement, R&D Systems, Minneapolis, Minn.).

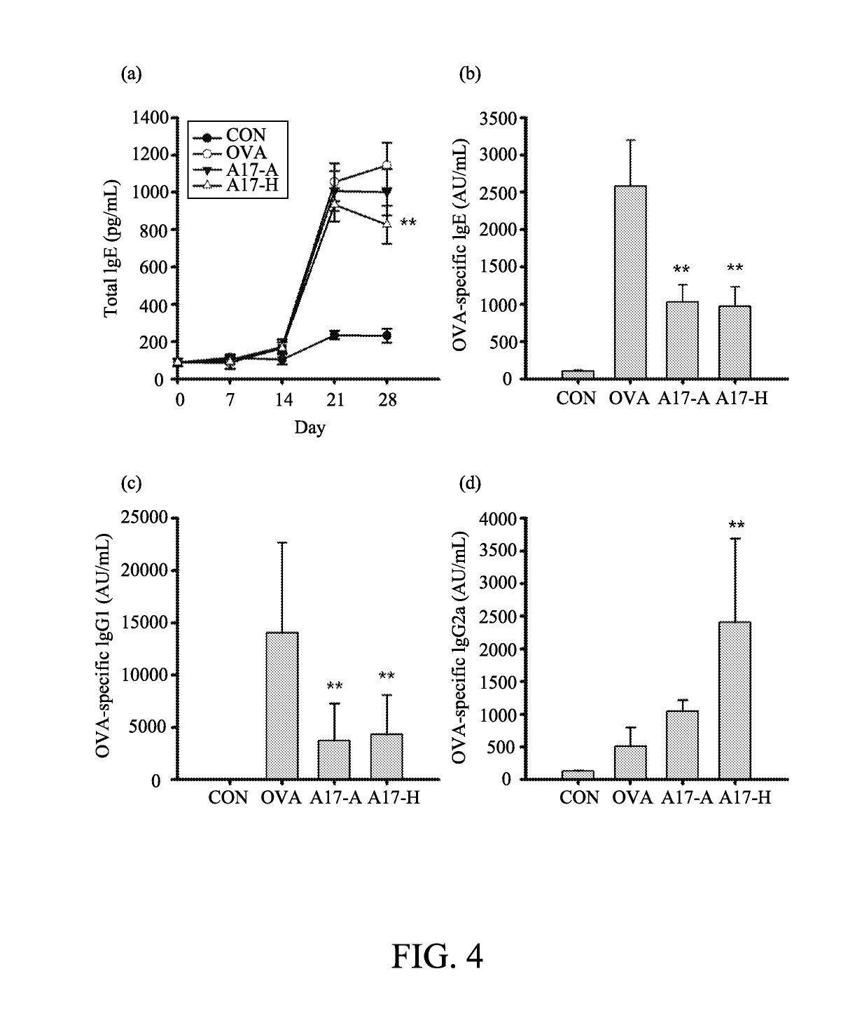

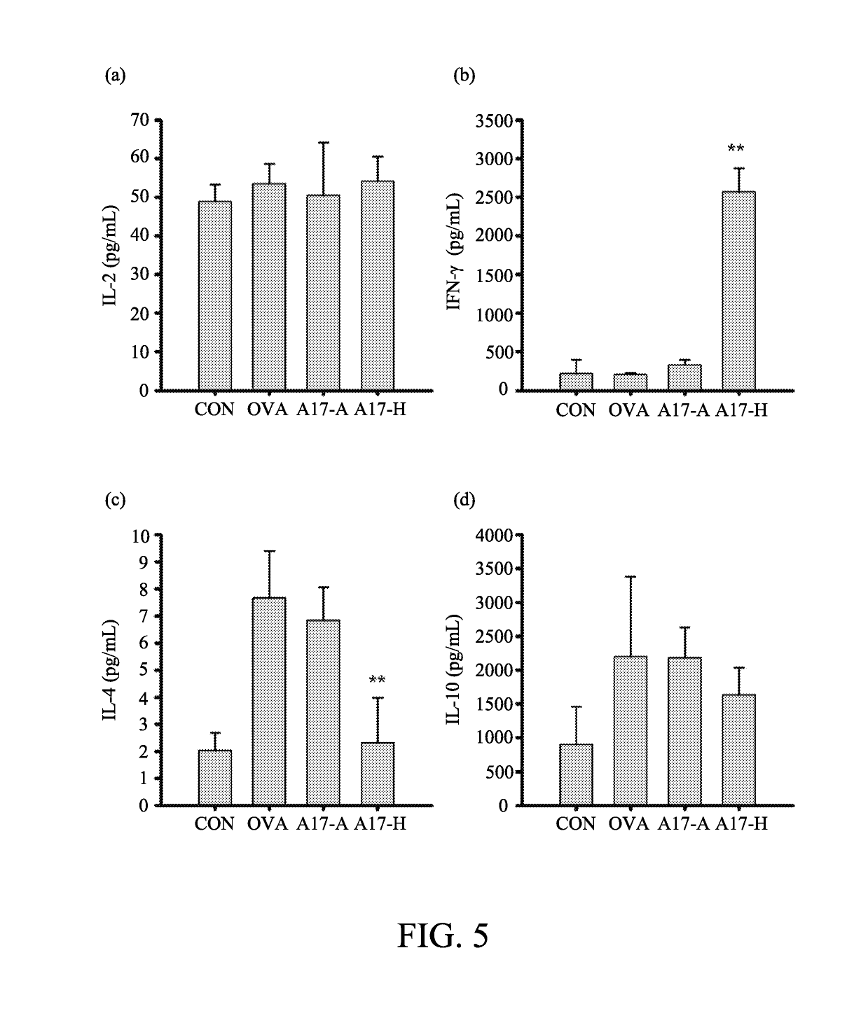

Some LAB strains with Th1 dominant responses are reported to be effective in regulating the production of OVA-induced immunoglobulins. In the present invention, the suppressive effects of A17 on immunoglobulin E production were analyzed as a preliminary experiment for B-cell response. As shown in FIG. 4(a), the total serum IgE in OVA-sensitized mice began elevated after day 14 and continuously increased through day 28. Oral administration of heat-killed A17 (A17-H) reduced the serum level of total IgE (FIG. 4(a)) and OVA-specific IgE (FIG. 4(b)) on day 28 compared to OVA-sensitized group. As for orally administered live A17 (A17-A), the level of OVA-specific IgE (FIG. 4(b)) was reduced. A17-H seemed to have a greater IgE suppressive effect than A17-A.

Furthermore, the serum levels of OVA-specific IgG1, Th2-type immunoglobulin, in the A17 groups (A17-A and A17-H) were significantly lower than that in the OVA-sensitized group (OVA) by about 3-fold (FIG. 4(c); P<0.01). The reduction in OVA-specific IgG1 amounts among heat-killed and live A17 treatment groups was comparable. In addition, A17-H significantly increased the serum level of OVA-specific IgG2a, the Th1-type immunoglobulin, compared with that in the OVA-sensitized group (OVA) (FIG. 4(d)). Apparently, both heat-killed and live A17 possessed the B-cell responsive ability to reduce Th2-type immunoglobulin production (such as IgE and IgG1) and induce Th1-type immunoglobulin production (such as IgG2a).

To evaluate the effects of live and heat-killed A17 supplementation on the T-cell responses, the concentrations of IFN-γ, IL-2, IL-4, and IL-10 in the supernatant of spleen cell cultures were measured (FIG. 5). Spleen cells from mice with OVA sensitization (OVA group and A17 groups) had no significant variation on IL-2 production as compared to the healthy control group (CON) (FIG. 5(a)). The level of IFN-γ in A17-H group was significantly elevated as compared to those in other groups (CON, OVA and A17-A groups) (FIG. 5(b)). As shown in FIG. 5(c), the IL-4 level of OVA-sensitized group (OVA) was significantly higher than that in the healthy control group (CON). While in A17 groups, the level of IL-4 in A17-H group was significantly lower than that in the OVA-sensitized group (OVA) and found to be similar to the healthy control group (CON). However, IL-4 production in A17-A was similar to OVA group. Furthermore, the level of IL-10, a regulatory cytokine, was also determined. When IL-10 was measured (FIG. 5(d)), it was found that the levels of IL-10 were elevated in OVA-sensitized (OVA) and A17 (A17-A and A17-H) groups. These results indicated that heat-killed A17 (A17-H) had a promising effect on modulating the T-cell responses in OVA-sensitized mice.

### Example 9

### Quantitative Real-Time RT-PCR

Total RNA from mouse spleen cells was prepared by using the TRIzol method (Invitrogen, Carlsbad, Calif.), and cDNA was then synthesized using the High Capacity cDNA Reverse Transcription Kit (ABI, Foster City, Calif.). Quantitative real-time PCR was performed in an ABI 7700 Real time PCR instrument according to the manufacturer's recommendations. Primer sets are listed in Table 4. The housekeeping gene glyceraldehyde-3 phosphate dehydrogenase (GAPDH) was used as an internal control. The expression levels of target mRNAs of each sample were normalized to GAPDH as an internal control.

TABLE 4
Primer Sets for the Real-Time RT-PCR

| Gene | | Size | Accession |
|---|---|---|---|
| name | Primer sequence | (bp) | no. |
| TLR-2 | F: GCTGGAGAACTCTGACCCGCC | | |
| | (SEQ ID NO. 5) | 217 | NM_011905.3 |
| | R: CAAGGATGGCCGCGTCGTTG | | |
| | (SEQ ID NO. 6) | | |
| TLR-4 | F: AGGAGTGCCCCGCTTTCACC | 203 | NM_021297.2 |
| | (SEQ ID NO. 7) | | |
| | R: TGCCAGAGCGGCTGCCAGA | | |
| | (SEQ ID NO. 8) | | |
| NOD-1 | F: AGCAGAACACCACACTGACA | 141 | NM_172729.3 |
| | (SEQ ID NO. 9) | | |
| | R: CCTTGGCTGTGATGCGAT | | |
| | (SEQ ID NO. 10) | | |
| NOD-2 | F: CAGGGACTCAAGAGCAACAC | 123 | NM_145857.2 |
| | (SEQ ID NO. 11) | | |
| | R: GCTGAGCCACTTTAGGTTCT | | |
| | (SEQ ID NO. 12) | | |
| GAPDH | F: GTATGACTCCACTCACGGCAAA | 101 | NM_008084 |
| | (SEQ ID NO. 13) | | |
| | R: GGTCTCGCTCCTGGAAGATG | | |
| | (SEQ ID NO. 14) | | |

F: forward primer;
R: reverse primer.

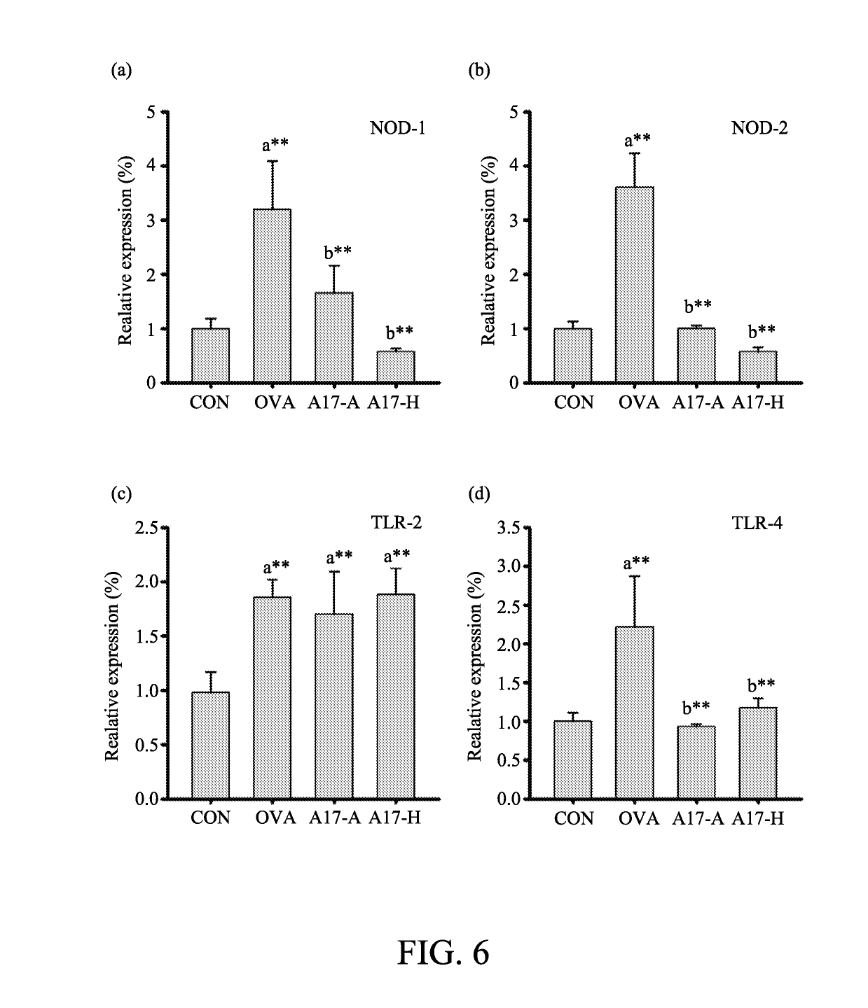

To evaluate the expression of TLR and NOD signaling in A17 orally administered mice, the splenic mRNA expression levels of NOD-1, NOD-2, TLR-2, and TLR-4, were examined by using real-time RT-PCR (FIG. 6). In the OVA group (OVA), the mRNA expression levels of NOD-1, NOD-2, TLR-2, and TLR-4 was elevated as compared to the healthy control group (CON)(P<0.01). When A17 (A17-A or A17-H) was orally administered to OVA-sensitized mice, the expression of NOD-1, NOD-2 and TLR-4 was significantly lower than that in OVA group (P<0.01). When NOD-1 and NOD-2 were observed, heat-killed A17 (A17-H) showed lower NOD-1 and NOD-2 expression levels than live A17 (A17-A). However, relative to the healthy control group, both A17-A and A17-H exhibited similar TLR-4 expression. The expression levels of TLR-2 were similar in the OVA group and A17 groups. These results indicated that OVA sensitization raised NOD-1, NOD-2, TLR-2, and TLR-4 in mouse spleen. In OVA-sensitized mice fed with live or heat-killed A17, the mRNA expression of NOD-1, NOD-2, and TLR-4 was diminished.

### Example 10

### Statistical Analysis

All data presented herein were expressed as means±the standard deviation (SD). The differences between means were tested for statistical significance using a one-way ANOVA followed by a Tukey's post-hoc test. Differences between the control group and other groups were considered statistically significant when the P<0.05 (*) or <0.01 (**).

In summary, as shown in FIG. 4, the increased levels of serum IgE, OVA-specific IgE, and IgG1 in the OVA group indicated a B-cell type Th2 responses. Both T-cell responsive Th2 cytokines IL-4 and IL-10 were also increased in the OVA group (FIG. 5). Moreover, the elevated mRNA expression of NOD-1 and NOD-2 in the OVA group represented an increase in Th2 responses (FIG. 6). In A17 groups, the levels of IgE, OVA-specific IgE, and OVA-specific IgG1 were significantly lower than those in the OVA group (P<0.01) (FIG. 4). Furthermore, a considerable increase of OVA-specific IgG2a was observed in the heat-killed A17 (A17-H) group (FIG. 4(d)). With regard to the cytokine production, The A17-H group showed a significantly higher level of IFN-γ and a lower IL-4 level relative to the OVA group (FIG. 5). Meanwhile, as shown in FIG. 6, the mRNA expression of NOD-1 and NOD-2 in both A17 groups was found to be significantly lower than that in the OVA group. Therefore, it suggested that the inhibitory effects of A17 on OVA-induced Th2 responses could be originated from down-regulation of NOD-1 and NOD-2 expression. In addition, it was found that the TLR-4 expression was elevated in the OVA group. After oral administration with both A17-A and A17-H, the TLR-4 expression was significantly diminished, compared to that in the OVA group (P<0.01). As such, it was further proved that the anti-allergic effects of A17 owed to the repression of NOD-1, NOD-2, and TLR-4 productions.

The foregoing descriptions of the detailed embodiments are only illustrated to disclose the principle and functions of the present invention and do not restrict the scope of the present invention. It should be understood to those in the art that all modifications and variations according to the spirit and principle in the disclosure of the present invention should fall within the scope of the appended claims. It is intended that the specification and examples are considered as exemplary only, with a true scope of the invention being indicated by the following claims.

The references listed below and the ATCC numbers cited in the application are each incorporated by reference as if they were incorporated individually.
[1] R. Bibiloni, R. N. Fedorak, G. W. Tannock, K. L. Madsen, P. Gionchetti, M. Campieri, C. De Simone, and R. B. Sartor, “VSL#3 probiotic-mixture induces remission in patients with active ulcerative colitis,” The American Journal of Gastroenterology, vol. 100, no. 7, pp. 1539-1546, 2005
[2] S. Rakoff-Nahoum, J. Paglino, F. Eslami-Varzaneh, S. Edberg, R. Medzhitov, “Recognition of commensal microflora by toll-like receptors is required for intestinal homeostasis,” Cell, vol. 118, no. 2, pp. 229-241, 2004.
[3] J. Ezendam, and H. van Loveren, “Probiotics: immunomodulation and evaluation of safety and efficacy,” Nutrition Reviews, vol. 64, no. 1, pp. 1-14, 2006.
[4] D. Fujiwara, S. Inoue, H. Wakabayashi, and T. Fujii, “The anti-allergic effects of lactic acid bacteria are strain dependent and mediated by effects on both Th1/Th2 cytokine expression and balance,” International Archives of Allergy and Immunology, vol. 135, no. 3, pp. 205-215, 2004.
[5] L. E. Niers, H. M. Timmerman, G. T. Rijkers et al., “Identification of strong interleukin-10 inducing lactic acid bacteria which down-regulate T helper type 2 cytokines,” Clinical and Experimental Allergy, vol. 35, no. 11, pp. 1481-1489, 2005.
[6] J. Wassenberg, S. Nutten, R. Audran et al., “Effect of Lactobacillus paracasei ST11 on a nasal provocation test with grass pollen in allergic rhinitis,” Clinical and Experimental Allergy, vol. 41, no. 4, pp. 565-573, 2011.
[7] T. J. Won, B. Kim, Y. T. Lim et al., “Oral administration of Lactobacillus strains from Kimchi inhibits atopic dermatitis in NC/Nga mice,” Journal of Applied Microbiology, vol. 110, no. 5, pp. 1195-1202, 2011.
[8] W. Eder, M. J. Ege, and von Mutius, E., “The asthma epidemic,” The New England Journal of Medicine, vol. 355, no. 21, pp. 2226-2235, 2006.
[9] M. Morisset, C. Aubert-Jacquin, P. Soulaines, D. A. Moneret-Vautrin, and C. Dupont, “A non-hydrolyzed, fermented milk formula reduces digestive and respiratory events in infants at high risk of allergy,” European Journal of Clinical Nutrition, vol. 65, no. 2, pp. 175-183, 2011.
[10] T. A. Platts-Mills, “The role of immunoglobulin E in allergy and asthma,” American Journal of Respiratory and Critical Care Medicine, vol. 164, no. 8 Pt 2, pp. S1-5, 2001.
[11] T. Morokata, J. Ishikawa, K. Ida, and T. Yamada, “C57BL/6 mice are more susceptible to antigen-induced pulmonary eosinophilia than BALB/c mice, irrespective of systemic T helper 1/T helper 2 responses,” Immunology, vol. 98, no. 3, pp. 345-351, 1999.
[12] M. Kalliomaki, S. Salmine, H. Arvilommi, P. Kero, P. Koskinen, E. Isolauri, “Probiotics in primary prevention of atopic disease: a randomised placebo-controlled trial,” Lancet, vol. 357, no. 9262, pp. 1076-1079. 2001.
[13] M. Kalliomaki, S. Salminen, T. Poussa, H. Arvilommi, E. Isolauri, “Probiotics and prevention of atopic disease: 4-year follow-up of a randomised placebo-controlled trial,” Lancet, vol. 361, no. 9372, pp. 1869-1871, 2003.
[14] K. Shida, K. Makino, A. Morishita et al., “Lactobacillus casei inhibits antigen-induced IgE secretion through regulation of cytokine production in murine splenocyte cultures,” International Archives of Allergy and Immunology, vol. 115, no. 4, pp. 278-287, 1998.
[15] K, Shida, R. Takahashi, E. Iwadate et al., “Lactobacillus casei strain Shirota suppresses serum immunoglobulin E and immunoglobulin G1 responses and systemic anaphylaxis in a food allergy model,” Clinical and Experimental Allergy, vol. 32, no. 4, pp. 563-570, 2002.
[16] Y. Inoue, N. Iwabuchi, J. Z. Xiao, T. Yaeshima, and K. Iwatsuki, “Suppressive effects of bifidobacterium breve strain M-16V on T-helper type 2 immune responses in a murine model,” Biological & Pharmaceutical Bulletin, vol. 32, no. 4, pp. 760-763, 2009.
[17] Y. G. Kim, J. H. Park, M. H. Shaw, L. Franchi, N. Inohara, and G. Nunez, “The cytosolic sensors Nod1 and Nod2 are critical for bacterial recognition and host defense after exposure to Toll-like receptor ligands,” Immunity, vol. 28, no. 2, pp. 246-257, 2008.
[18] J. R. Li, and Y. H. Hsieh, “Traditional Chinese food technology and cuisine,” Asia Pacific Journal of Clinical Nutrition, vol. 13, no. 2, pp. 147-155, 2004.
[19] S. H. Chao, Y. Tomii, K. Watanabe, and Y. C. Tsai, “Diversity of lactic acid bacteria in fermented brines used to make stinky tofu,” International Journal of Food Microbiology, vol. 123, no. 1-2, pp. 134-141, 2008.
[20] S. H. Chao, R. J. Wu, K. Watanabe, and Y. C. Tsai, “Diversity of lactic acid bacteria in suan-tsai and fu-tsai, traditional fermented mustard products of Taiwan,” International Journal of Food Microbiology, vol. 135, no. 3, pp. 203-210, 2009.
[21] Y. W. Liu, J. C. Liu, C. Y. Huang, C. K. Wang, H. F. Shang, and W. C. Hou, “Effects of oral administration of yam tuber storage protein, dioscorin, to BALB/c mice for 21-days on immune responses,” Journal of Agricultural and Food Chemistry, vol. 57, no. 19, pp. 9274-9279, 2009.
[22] T. Matsuzaki, “Immunomodulation by treatment with Lactobacillus casei strain Shirota,” International Journal of Food Microbiology, vol. 41, no. 2, pp. 133-140, 1998.
[23] Y. W. Liu, H. F. Shang, C. K. Wang, F. L. Hsu, and W. C. Hou, “Immunomodulatory activity of dioscorin, the storage protein of yam (Dioscorea alata cv. Tainong No. 1) tuber,” Food and Chemical Toxicology, vol. 45, no. 11, pp. 2312-2318, 2007.
[24] Y. Sato, H. Akiyama, H. Suganuma et al., “The feeding of beta-carotene down-regulates serum IgE levels and inhibits the type I allergic response in mice,” Biological & Pharmaceutical Bulletin, vol. 27, no. 7, pp. 978-984, 2004.
[25] Y. W. Liu, Y. W. Su, W. K. Ong, T. H. Cheng, and Y. C. Tsai, “Oral administration of Lactobacillus plantarum K68 ameliorates DSS-induced ulcerative colitis in BALB/c mice via the anti-inflammatory and immunomodulatory activities,” International Immunopharmacology, vol. 11, no. 12, pp. 2159-2166, 2011.
[26] H. Yasui, J. Kiyoshima, and T. Hori, “Reduction of influenza virus titer and protection against influenza virus infection in infant mice fed Lactobacillus casei Shirota,” Clinical and Diagnostic Laboratory Immunology, vol. 11, no. 4, pp. 675-679, 2004.
[27] C. C. Chen, W. C. Lin, M. S. Kong et al., “Oral inoculation of probiotics Lactobacillus acidophilus NCFM suppresses tumour growth both in segmental orthotopic colon cancer and extra-intestinal tissue,” The British Journal of Nutrition, vol. 30, no. pp. 1-12, 2011.
[28] L. G. Bermudez-Humaran, N. G. Cortes-Perez, F. Lefevre et al., “A novel mucosal vaccine based on live Lactococci expressing E7 antigen and IL-12 induces systemic and mucosal immune responses and protects mice against human papillomavirus type 16-induced tumors,” Journal of Immunology, vol. 175, no. 11, pp. 7297-7302, 2005.
[29] A. Miyoshi, I. Poquet, V. Azevedo et al., “Controlled production of stable heterologous proteins in Lactococcus lactis,” Applied and Environmental Microbiology, vol. 68, no. 6, pp. 3141-3146, 2002.
[30] M. Bahey-El-Din, C. G. Gahan, and B. T. Griffin, “Lactococcus lactis as a cell factory for delivery of therapeutic proteins,” Current Gene Therapy, vol. 10, no. 1, pp. 34-45, 2010.
[31] N. G. Cortes-Perez, S. Ah-Leung, L. G. Bermudez-Humaran et al., “Intranasal coadministration of live lactococci producing interleukin-12 and a major cow's milk allergen inhibits allergic reaction in mice,” Clinical and Vaccine Immunology, vol. 14, no. 3, pp. 226-233, 2007.
[32] B. Marelli, A. R. Perez, C. Banchio, D. de Mendoza, and C. Magni, “Oral immunization with live Lactococcus lactis expressing rotavirus VP8 subunit induces specific immune response in mice,” Journal of Virological Methods, vol. 175, no. 1, pp. 28-37, 2011.
[33] J. Debarry, H. Garn, A. Hanuszkiewicz et al., “Acinetobacter lwoffii and Lactococcus lactis strains isolated from farm cowsheds possess strong allergy-protective properties,” The Journal of Allergy and Clinical Immunology, vol. 119, no. 6, pp. 1514-1521, 2007.
[34] P. Rupa, J. Schmied, and B. N. Wilkie, “Prophylaxis of experimentally induced ovomucoid allergy in neonatal pigs using Lactococcus lactis,” Veterinary Immunology and Immunopathology, vol. 140, no. 1-2, pp. 23-29, 2011.
[35] K. Shida, J. Kiyoshima-Shibata, M. Nagaoka, K. Watanabe, and M. Nanno, “Induction of interleukin-12 by Lactobacillus strains having a rigid cell wall resistant to intracellular digestion,” Journal of Dairy Science, vol. 89, no. 9, pp. 3306-3317, 2006.
[36] A. Inamine, D. Sakurai, S. Horiguchi et al., “Sublingual administration of Lactobacillus paracasei KW3110 inhibits Th2-dependent allergic responses via upregulation of PD-L2 on dendritic cells,” Clinical Immunology, vol. 143, no. 2, pp. 170-179, 2012.
[37] J. D. Greene, and T. R. Klaenhammer, “Factors involved in adherence of lactobacilli to human Caco-2 cells,” Applied and Environmental Microbiology, vol. 60, no. 12, pp. 4487-4494, 1994.
[38] A. C. Ouwehand, E. Isolauri, P. V. Kirjavainen, and S. J. Salminen, “Adhesion of four Bifidobacterium strains to human intestinal mucus from subjects in different age groups,” FEMS Microbiology Letters, vol. 172, no. 1, pp. 61-64, 1999.
[39] E. J. Schiffrin, D. Brassart, A. L. Servin, F. Rochat, and A. Donnet-Hughes, “Immune modulation of blood leukocytes in humans by lactic acid bacteria: criteria for strain selection,” The American Journal of Clinical Nutrition, vol. 66, no. 2, pp. 515S-520S, 1997.
[40] Y. Nonaka, T. Izumo, F. Izumi et al., “Antiallergic effects of Lactobacillus pentosus strain S-PT84 mediated by modulation of Th1/Th2 immunobalance and induction of IL-10 production,” International Archives of Allergy and Immunology, vol. 145, no. 3, pp. 249-257, 2008.
[41] A. Yoshida, R. Aoki, H. Kimoto-Nira et al., “Oral administration of live Lactococcus lactis C59 suppresses IgE antibody production in ovalbumin-sensitized mice via the regulation of interleukin-4 production,” FEMS Immunology and Medical Microbiology, vol. 61, no. 3, pp. 315-322, 2011.
[42] E. Meylan, J. Tschopp, and M. Karin, “Intracellular pattern recognition receptors in the host response,” Nature, vol. 442, no. 7098, pp. 39-44, 2006.
[43] K. Takeda, T. Kaisho, and S. Akira, “Toll-like receptors,” Annual Review of Immunology, vol. 21, no. pp. 335-376, 2003.
[44] W. Duan, A. K. Mehta, J. G. Magalhaes et al., “Innate signals from Nod2 block respiratory tolerance and program T(H)2-driven allergic inflammation,” The Journal of Allergy and Clinical Immunology, vol. 126, no. 6, pp. 1284-1293 e1210, 2010.
[45] J. G. Magalhaes, S. J. Rubino, L. H. Travassos et al., “Nucleotide oligomerization domain-containing proteins instruct T cell helper type 2 immunity through stromal activation,” Proceedings of The National Academy of Sciences of The United States of America, vol. 108, no. 36, pp. 14896-14901, 2011.
[46] S. C. Eisenbarth, D. A. Piggott, J. W. Huleatt, I. Visintin, C. A. Herrick, and K. Bottomly, “Lipopolysaccharide-enhanced, toll-like receptor 4-dependent T helper cell type 2 responses to inhaled antigen,” The Journal of Experimental Medicine, vol. 196, no. 12, pp. 1645-1651, 2002.
[47] V. Redecke, H. Hacker, S. K. Datta et al., “Cutting edge: activation of Toll-like receptor 2 induces a Th2 immune response and promotes experimental asthma,” Journal of Immunology, vol. 172, no. 5, pp. 2739-2743, 2004.
[48] M. Schnare, G. M. Barton, A. C. Holt, K. Takeda, S. Akira, and R. Medzhitov, “Toll-like receptors control activation of adaptive immune responses,” Nature Immunology, vol. 2, no. 10, pp. 947-950, 2001.
[49] C. A. Salkowski, G. R. Detore, and S. N. Vogel, “Lipopolysaccharide and monophosphoryl lipid A differentially regulate interleukin-12, gamma interferon, and interleukin-10 mRNA production in murine macrophages,” Infection and Immunity, vol. 65, no. 8, pp. 3239-3247, 1997.
[50] A. Yoshida, and Y. Koide, “Arabinofuranosyl-terminated and mannosylated lipoarabinomannans from Mycobacterium tuberculosis induce different levels of interleukin-12 expression in murine macrophages,” Infection and Immunity, vol. 65, no. 5, pp. 1953-1955, 1997.
[51] M. G. Cleveland, J. D. Gorham, T. L. Murphy, E. Tuomanen, and K. M. Murphy, “Lipoteichoic acid preparations of gram-positive bacteria induce interleukin-12 through a CD 14-dependent pathway,” Infection and Immunity, vol. 64, no. 6, pp. 1906-1912, 1996.
[52] E. Lien, T. J. Sellati, A. Yoshimura et al., “Toll-like receptor 2 functions as a pattern recognition receptor for diverse bacterial products,” The Journal of Biological Chemistry, vol. 274, no. 47, pp. 33419-33425, 1999.
[53] M. D. Halpern, R. J. Kurlander, and D. S. Pisetsky, “Bacterial DNA induces murine interferon-gamma production by stimulation of interleukin-12 and tumor necrosis factor-alpha,” Cellular Immunology, vol. 167, no. 1, pp. 72-78, 1996.
[54] K. Takeda, and S. Akira, “Toll-like receptors in innate immunity,” International Immunology, vol. 17, no. 1, pp. 1-14, 2005.
[55] Y. H. Ryu, J. E. Baik, J. S. Yang et al., “Differential immunostimulatory effects of Gram-positive bacteria due to their lipoteichoic acids,” International Immunology, vol. 9, no. 1, pp. 127-133, 2009.
[56] T. Sashihara, N. Sueki, and S. Ikegami, “An analysis of the effectiveness of heat-killed lactic acid bacteria in alleviating allergic diseases,” Journal of Dairy Science, vol. 89, no. 8, pp. 2846-2855, 2006.
[57]C. C. Ou, S. L. Lin, J. J. Tsai, and M. Y. Lin, “Heat-killed lactic acid bacteria enhance immunomodulatory potential by skewing the immune response toward Th1 polarization,” Journal of Food Science, vol. 76, no. 5, pp. M260-267, 2011.
[58] Chao, S. H., Tomii, Y., Watanabe, K., Tsai, Y. C. 2008. Diversity of lactic acid bacteria in fermented brines used to make stinky tofu. International Journal of Food Microbiology. 123: 134-141.
[59] Maidak, B. L., Cole, J. R., Parker, C. T. Jr., Garrity, G. M., Larsen, N., Li, B., Lilburn, T. G, McCaughey, M. J., Olsen, G. J., Overbeek, R., Pramanik, S., Schmidt, T. M., Tiedje, J. M. & Woese, C. R. (1999). A new version of the RDP (Ribosomal Database Project). Nucl. Acids Res. 27, 171-173.
[60]. Akopyanz N, Bukanov N O, Westblom T U, Kresovich S, Berg D E. DNA diversity among clinical isolates of Helicobacter pylori detected by PCR-based RAPD fingerprinting. Nucleic Acids Res. 1992 Oct. 11; 20(19):5137-42

1. A lactic acid bacterium being Lactococcus lactis subsp. cremoris A17 and deposited under DSMZ Accession No. DSM 27109, wherein the lactic acid bacterium is heat-inactivated.

2. (canceled)

3. A composition, comprising the lactic acid bacterium of claim 1 and a carrier.

4. (canceled)

5. A method for treating or preventing a disorder in a subject, comprising:
administering an effective amount of a lactic acid bacterium according to claim 1 to the subject.

6. The method according to claim 5, wherein the lactic acid bacterium is heat-inactivated.

7. The method according to claim 5, wherein the disorder is related to expression of a protein selected from the group consisting of IgG1, IgG2a, IgE, IFN-γ, IL-4, NOD-1, NOD-2 and TLR-4.

8. The method according to claim 7, wherein the expression of IgG2a or IFN-γ is increased.

9. The method according to claim 7, wherein the expression of IgG1, IgE or IL-4 is decreased.

10. The method according to claim 7, wherein mRNA expression of NOD-1, NOD-2 or TLR-4 is down-regulated.

11. The method according to claim 5, wherein the disorder is an allergic disorder.

12. The method according to claim 11, wherein the allergic disorder is allergic rhinitis, atopic dermatitis, allergic asthma or a food allergy.

13. The method according to claim 5, wherein the lactic acid bacterium is orally administrated.

14. A method for modulating an immune response in a subject, comprising:
administering an effective amount of a lactic acid bacterium according to claim 1 to the subject.

15. The method according to claim 14, wherein the lactic acid bacterium is heat-inactivated.

16. The method according to claim 14, wherein the immune response is related to expression of a protein selected from the group consisting of IgG1, IgG2a, IgE, IFN-γ, IL-4, NOD-1, NOD-2 and TLR-4.

17. The method according to claim 16, wherein the expression of IgG2a or IFN-γ is increased.

18. The method according to claim 16, wherein the expression of IgG1, IgE or IL-4 is decreased.

19. The method according to claim 16, wherein mRNA expression of NOD-1, NOD-2 or TLR-4 is down-regulated.

20. The method according to claim 14, wherein the immune response is related to an allergic disorder.

21. The method according to claim 20, wherein the allergic disorder is allergic rhinitis, atopic dermatitis, allergic asthma or a food allergy.

22. The method according to claim 14, wherein the lactic acid bacterium is orally administrated.
